(19) United States
(12) Patent Application Publication
(10) Pub. No.: US 2026/0137610 A1
(43) Pub. Date: May 21, 2026

(54) SHAMPOO GRANULE AND PREPARATION METHOD THEREOF

(71) Applicant: Guangzhou Beaver Cosmetics Co., Ltd., Guangzhou (CN)

(72) Inventor: Hongpeng Zhang, Guangzhou (CN)

(21) Appl. No.: 19/241,396

(22) Filed: Jun. 18, 2025

(30) Foreign Application Priority Data
Nov. 20, 2024 (CN) 202411661381.8

(51) Int. Cl. A61K 8/46; A61K 8/44; A61K 8/73; A61K 8/92; A61K 8/9794; A61K 8/99; A61Q 5/02

(52) U.S. Cl. CPC A61K 8/466; A61K 8/44; A61K 8/732; A61K 8/737; A61K 8/922; A61K 8/9794; A61K 8/99; A61Q 5/02

(57) ABSTRACT

A shampoo granule and a preparation method thereof are provided. The shampoo granule includes the following components in parts by weight: 0.0008-0.0072 parts of a softener, 53-92 parts of a surfactant, 5-25 parts of an adsorbent, 1-10 parts of a moisturizer, 0.5-6 parts of an anti-caking agent, 0.4-5 parts of a hair regulator, 0.1-1 part of a pH regulator, 0.2-1.4 parts of a preservative, 0.1-1 part of a thickener and 0.5-1.5 parts of a fragrance; where the surfactant is a mixture of sodium cocoyl hydroxyethylsulfonate and sodium lauryl glutamate; the softener is a mixture of acanthus kernel oil, avocado fruit oil, grapeseed oil and Lactobacillus/rye flour ferment filtrate. The shampoo granule has excellent gentleness and nourishing cleansing efficacy, and is easy to rinse without residue, which can significantly reduce water consumption during use. The shampoo granule also has advantages of easy transportation and carrying.

### CROSS-REFERENCE TO RELATED APPLICATION

This disclosure claims the benefit of Chinese patent application No. 202411661381.8, filed on Nov. 20, 2024, which is incorporated herein by reference in its entirety.

### TECHNICAL FIELD

This disclosure belongs to the technical field of daily chemical products, and in particular relates to a shampoo granule and a preparation method thereof.

### BACKGROUND

At present, traditional liquid shampoo is widely used in the market, but there are some problems that cannot be ignored. Firstly, many consumers are prone to scalp problems such as itching and increased dandruff after using liquid shampoos. This is usually because the liquid shampoo contains a large amount of chemicals such as surfactants and thickeners, which may irritate sensitive scalp, leading to excessive dryness or allergic reactions of the scalp, which in turn lead to scalp discomfort and dandruff production. Secondly, the allergy problem brought by the liquid shampoos is also common. Some shampoos contain a large amount of chemicals such as pigments and preservatives, which may cause scalp allergic reactions. Especially for consumers with sensitive skin, the liquid shampoo may cause scalp redness, swelling, itching and other symptoms. Thirdly, the production process of liquid shampoos is also facing the problems of environmental protection and resource consumption. The production of liquid shampoos requires a large amount of water, and due to complex formulations and multiple packaging of the liquid shampoos, the waste of water resources and other resources is quite severe. Water consumption in the production process, the weight of the liquid shampoos during transportation and the waste of water during use will put pressure on water resources, and also increase the carbon footprint and environmental burden.

Therefore, developing a solid shampoo granule that can gently clean hair and is easy to rinse without residue becomes a worthy alternative. The shampoo granule not only reduces scalp irritation and allergic reaction, but also reduces water consumption. Further, it has environmental advantages and is easy to transport or carry. Therefore, it is in line with the modern consumer's dual needs for both environmental protection and health, and can fill the market gaps.

### SUMMARY

An objective of this disclosure is to overcome the shortcomings of the prior art and provide a shampoo granule and a preparation method thereof.

To achieve the above objective, the following technical solutions are adopted.

In a first aspect, provided herein is a shampoo granule, wherein the shampoo granule comprises the following components in parts by weight: 0.0008-0.0072 parts of a softener, 53-92 parts of a surfactant, 5-25 parts of an adsorbent, 1-10 parts of a moisturizer, 0.5-6 parts of an anti-caking agent, 0.4-5 parts of a hair regulator, 0.1-1 part of a pH regulator, 0.2-1.4 parts of a preservative, 0.1-1 part of a thickener and 0.5-1.5 parts of a fragrance;
the surfactant is a mixture of sodium cocoyl hydroxyethylsulfonate and sodium lauryl glutamate; and
the softener is a mixture of acanthus kernel oil, avocado fruit oil, grapeseed oil and Lactobacillus/rye flour ferment filtrate.

In this disclosure, sodium cocoyl hydroxyethylsulfonate and sodium lauroyl glutamate are blended as a surfactant. The two cooperate with each other, to make the shampoo granule achieve lower irritability and good cleansing efficacy. When the shampoo granule is used for washing hair, excessive degreasing, over-drying or allergic phenomena can be avoided. In addition, the two have excellent easy rinsing property, which ensures that no residue is left after the hair is washed by using the shampoo granule, ensuring that the hair is fresh and smooth after washing, and improving the consumer's experience.

In addition, the acanthus kernel oil, the avocado fruit oil, the grapeseed oil and the Lactobacillus/rye flour ferment filtrate are combined with the surfactant in this disclosure, which can further enhance the multiple hair care and cleansing efficacy of the shampoo granule, thereby providing excellent gentleness, nourishing efficacy and easy rinsing property, and ensuring the scalp is fresh and comfortable. Therefore, the shampoo granule is suitable for all kinds of hair, especially for consumers with sensitive scalp. The argan kernel oil is rich in essential fatty acids, vitamin E and antioxidants, which can provide deep nutrition for hair and scalp, and help remove oil and dirt from hair; the avocado oil is rich in monounsaturated fatty acids and vitamins A, D, E and K, which can deeply nourish hair and scalp, and help relieve problems such as dry scalp, itching and dandruff; the grapeseed oil is rich in polyphenols and antioxidants, which can enhance hair health and prevent damage brought by free radicals, and is conductive to regulation of sebum secretion of the scalp and keeping hair fresh; and the Lactobacillus/rye flour ferment filtrate is rich in natural lactic acid and beneficial microbial components, which can regulate microecology of scalp and maintain pH balance of scalp.

Through experimental research, it has been found that the four softener components are blended as a softener, which is further combined with other components such as the specified surfactant for the preparation of the shampoo granule in this disclosure, the four softener components cooperate with one another, which can make the shampoo granule achieve more gentle and excellent cleansing and decontamination efficacy. Meanwhile, the four softener components undergo affinity reactions with the surfactant, so as to form a more uniform and more stable granular product.

Preferably, the shampoo granule comprises the following components in parts by weight: 0.0015-0.0045 parts of the softener, 62-82 parts of the surfactant, 10-20 parts of the adsorbent, 3-8 parts of the moisturizer, 1-5 parts of the anti-caking agent, 1-3 parts of the hair regulator, 0.2-0.8 parts of the pH regulator, 0.4-1.2 parts of the preservative, 0.2-0.8 parts of the thickener and 0.7-1.3 parts of the fragrance.

More preferably, the shampoo granule comprises the following components in parts by weight: 0.004 parts of the softener, 75 parts of the surfactant, 20 parts of the adsorbent, 5 parts of the moisturizer, 3 parts of the anti-caking agent, 2 parts of the hair regulator, 0.5 parts of the pH regulator, 0.8 parts of the preservative, 0.5 parts of the thickener and 1 part of the fragrance.

Preferably, a mass ratio of sodium cocoyl hydroxyethylsulfonate to sodium lauryl glutamate is (57-67):(5-15).

More preferably, the mass ratio of sodium cocoyl isethionate to sodium lauryl glutamate is 60:15.

Preferably, a mass ratio of the acanthus kernel oil:the avocado fruit oil:the grapeseed oil:the Lactobacillus/rye flour ferment filtrate is (0.0005-0.0015):(0.0005-0.0015):(0.0005-0.0015):(0.0005-0.0015).

Preferably, the adsorbent comprises a modified corn starch, the moisturizer comprises betaine, the hair regulator comprises guar gum hydroxypropyl trimethyl ammonium chloride, and the thickener comprises hydroxypropyl methyl cellulose.

Preferably, the anti-caking agent comprises magnesium stearate, the pH regulator comprises citric acid, the preservative comprises chlorphenesin and/or benzyl alcohol, and the fragrance comprises an essence.

Preferably, the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules within a 10-100 mesh range, to give the shampoo granule.

Preferably, in the step S2, a mass ratio of the powder to the thickener aqueous solution is 1:0.3.

The specified method is adopted in this disclosure to prepare the shampoo granule, in which all components in the granule are evenly mixed, avoiding the separation or non-uniformity distribution of components in the final product, which is conductive to improving the stability and consistency of the shampoo granule. Further, the size distribution of the granules can be effectively controlled and the uniformity and hardness of the granules are ensured, thereby achieving good solubility and washing efficacy of the product. In addition, the solid shampoo granule innovatively developed in this disclosure has low water activity, which can effectively inhibit the activities of bacteria and other microorganisms, reduce the deterioration risk of the product, reduce the amount of preservatives, and improve the stability of the active ingredients in the granule. Furthermore, the low water activity is conductive to controlling the generation of foam, so as to achieve easy rinsing efficacy without residue. The water consumption during use is significantly reduced. Compared with traditional liquid shampoos, the present shampoo granule has a greatly reduced volume, and it is safer, more efficient and convenient in the transportation process.

Compared with the prior art, the disclosure has the following beneficial effects:

In this disclosure, a shampoo granule is innovatively developed by combining the specified surfactant and the softener with other components through a specified preparation method. The components of the shampoo granule cooperate with one another, to play multiple hair care and cleansing efficacy, thereby providing excellent gentle and nourishing effects, and ensuring that the scalp is fresh and comfortable after washing hair with the shampoo granule. Moreover, compared with the traditional liquid shampoos, the shampoo granule of this disclosure is easy to rinse without residue, which can significantly reduce the water consumption during use and reduce the consumption of water resources. In addition, the shampoo granule has a greatly reduced volume, and is easy to transport or carry. Therefore, the shampoo granule is in line with the modern consumer's dual needs for both environmental protection and health.

### DETAILED DESCRIPTION

To better explain the objectives, technical solutions and advantages of the disclosure, specific examples are provided to further explain the disclosure. It should be understood by those skilled in the art that the specific examples are provided only to explain the disclosure, and are not intended to limit it.

Raw materials involved in the following examples and comparative examples are commercially available materials, unless otherwise specified.

### Examples 1-5

The shampoo granules were provided in Examples 1-5, and the specific formulas were shown in Table 1. The preparation method of the shampoo granules comprised the following steps.
S1, a surfactant, an adsorbent, a moisturizer, an anti-caking agent, a hair regulator, a fragrance, a pH regulator, a preservative and a softener were mixed evenly to give a mixture, and the mixture was ground into powder;
S2, a thickener was mixed with water to give a thickener aqueous solution with a mass concentration of 0.5%, the powder obtained in step S1 was added to a granulator, then the thickener aqueous solution was sprayed into the granulator by a spraying device to give granules, and then the granules were dried at a high temperature;
S3, granules within a 10-100 mesh range were sieved, to give the shampoo granule.

In step S2, a mass ratio of the powder to the thickener aqueous solution was 1:0.3, that is, 1 kg of powder was used with 0.3 kg of the thickener aqueous solution.

TABLE 1
Formulas (components in parts by weight) of the shampoo granules in Examples 1-5

| | | | Example | Example | Example | Example |
|---|---|---|---|---|---|---|
| | Component | Example 1 | 2 | 3 | 4 | 5 |
| Surfactant | Sodium cocoyl | 60 | 52 | 72 | 57 | 67 |
| | hydroxyethylsulfonate | | | | | |
| Adsorbent | Modified corn starch | 20 | 5 | 25 | 10 | 20 |
| Surfactant | Sodium lauryl glutamate | 15 | 1 | 20 | 5 | 15 |
| Moisturizer | Betaine | 5 | 1 | 10 | 3 | 8 |
| Anti-caking | Magnesium stearate | 3 | 0.5 | 6 | 1 | 5 |
| agent | | | | | | |
| Hair | Guar gum hydroxypropyl | 2 | 0.5 | 4 | 1 | 3 |
| regulator | trimethyl ammonium | | | | | |
| | chloride | | | | | |
| pH regulator | Citric acid | 0.5 | 0.1 | 1 | 0.2 | 0.8 |
| Preservative | Chlorphenesin | 0.2 | 0 | 0.4 | 0.1 | 0.3 |
| Fragrance | (daily) essence | 1 | 0.5 | 1.5 | 0.7 | 1.3 |
| Preservative | Benzyl alcohol | 0.6 | 0.2 | 1 | 0.3 | 0.9 |
| Softener | Lactobacillus/rye flour | 0.001 | 0.0002 | 0.0018 | 0.0005 | 0.0015 |
| | ferment filtrate | | | | | |
| | Acanthus kernel oil | 0.001 | 0.0002 | 0.0018 | 0.0005 | 0.0015 |
| | Grapeseed oil | 0.001 | 0.0002 | 0.0018 | 0.0005 | 0.0015 |
| | Avocado fruit oil | 0.001 | 0.0002 | 0.0018 | 0.0005 | 0.0015 |
| Thickener | Hydroxypropyl methyl | 0.5 | 0.1 | 1 | 0.2 | 0.8 |
| | cellulose | | | | | |

### Comparative Examples 1-7

The shampoo granules were provided in Comparative Examples 1-7, and the specific formulas were shown in Table 2. The preparation method of each of the shampoo granules of Comparative Examples 1-7 was similar to that of Examples 1-5, except that the formulas were different.

TABLE 2
Formulas (components in parts by weight) of the shampoo granules in Comparative Examples 1-7

| | Component | CEx. 1 | CEx. 2 | CEx. 3 | CEx. 4 | CEx. 5 | CEx. 6 | CEx. 7 |
|---|---|---|---|---|---|---|---|---|
| Surfactant | Sodium cocoyl | / | 60 | 45 | 80 | 60 | 60 | 60 |
| | hydroxyethylsulfonate | | | | | | | |
| Adsorbent | Modified corn starch | 20 | 20 | 20 | 20 | 20 | 20 | 20 |
| Surfactant | Sodium lauryl | 15 | / | 0.5 | 25 | 15 | 15 | / |
| | glutamate | | | | | | | |
| Moisturizer | Betaine | 5 | 5 | 5 | 5 | 5 | 5 | 5 |
| anti-caking | Magnesium stearate | 3 | 3 | 3 | 3 | 3 | 3 | 3 |
| agent | | | | | | | | |
| Hair | Guar gum | 2 | 2 | 2 | 2 | 2 | 2 | 2 |
| regulator | hydroxypropyl | | | | | | | |
| | trimethyl ammonium | | | | | | | |
| | chloride | | | | | | | |
| pH regulator | Citric acid | 0.5 | 0.5 | 0.5 | 0.5 | 0.5 | 0.5 | 0.5 |
| Preservative | Chlorphenesin | 0.2 | 0.2 | 0.2 | 0.2 | 0.2 | 0.2 | 0.2 |
| Fragrance | (daily) essence | 1 | 1 | 1 | 1 | 1 | 1 | 1 |
| Preservative | Benzyl alcohol | 0.6 | 0.6 | 0.6 | 0.6 | 0.6 | 0.6 | 0.6 |
| Softener | Lactobacillus/rye | 0.001 | 0.001 | 0.001 | 0.001 | / | 0.001 | / |
| | flour ferment filtrate | | | | | | | |
| | Acanthus kernel oil | 0.001 | 0.001 | 0.001 | 0.001 | 0.001 | / | / |
| | Grapeseed oil | 0.001 | 0.001 | 0.001 | 0.001 | / | 0.001 | 0.001 |
| | Avocado fruit oil | 0.001 | 0.001 | 0.001 | 0.001 | 0.001 | / | 0.001 |
| Thickener | Hydroxypropyl | 0.5 | 0.5 | 0.5 | 0.5 | 0.5 | 0.5 | 0.5 |
| | methyl cellulose | | | | | | | |

Note:
CEx. represents “Comparative Example”.

### Comparative Example 8

A liquid shampoo was provided in this comparative example, which differed from Example 1 only in the preparation method. All components and amounts thereof were the same as those in Example 1.

The preparation method of the liquid shampoo in this comparative example comprised the following steps.

A surfactant, an adsorbent, a moisturizer, an anti-caking agent, a hair regulator, a fragrance, a pH regulator, a preservative, a softener and a thickener were added to water, and stirred well at 40° C. to give a thick shampoo in a form of emulsion.

### Effect Example

### 1. Water Activity (Aw) Test:

The shampoo granules prepared in Example 1 and Comparative Examples 1-4 were each taken as a sample, and tested for water activity. The test method was carried out using a water activity meter, according to United States Pharmacopeia—National Formulary (USP-NF) General Chapter 922: Water Activity. The results were shown in Table 3.

TABLE 3

| Sample | Water activity |
|---|---|
| Example 1 | 0.31 |
| Comparative Example 1 | 0.82 |
| Comparative Example 2 | 0.65 |
| Comparative Example 3 | 0.58 |
| Comparative Example 4 | 0.44 |

Water activity (Aw) refers to the availability or activity of water in a substance, which measures the degree to which water can participate in biochemical reactions in a system. In the preparation process of a shampoo granule, water activity has an important impact on the properties, stability and use effect of the product. In general, a shampoo granule with a lower water activity is more conducive to improving the stability of the product, which avoids the degradation or inefficiency of the active ingredients and effectively inhibits the growth and reproduction of microorganisms, thereby reducing the deterioration risk of the product. In addition, reduced water activity helps to control the generation of foam and achieve a more stable amount and quality of foam, so that the shampoo granule can generate more uniform foam which is easy to rinse without residue.

As can be seen from the results in Table 3, the water activity of the shampoo granule obtained in this disclosure reaches 0.31, which is significantly lower than those in the comparative examples. However, in Comparative Examples 1-2, sodium cocoyl hydroxyethylsulfonate or sodium lauryl glutamate is absent in the surfactant, the water activity of the shampoo granule is increased compared with that of Example 1. This might be because the absence of one of the two surfactant components causes the other components in the granule to not combine fully, resulting in an unstable granular structure and an increase of the water activity of the granule. In Comparative Examples 3-4, the mass ratio of sodium cocoyl hydroxyethylsulfonate to sodium lauryl glutamate exceeds the range specified in this disclosure, the water activity of the resulting shampoo granules also decreases, and the foam performance, storage stability and usage efficacy of the shampoo granule may be affected.

### 2. Stimulation Test:

The shampoo granules prepared in Examples 1-5 and Comparative Examples 1-4, and the liquid shampoo prepared in Comparative Example 8 were each taken as a sample, and tested for skin irritation. The test method was as follows.

A total of 39 healthy adult rabbits were randomly selected and divided into thirteen groups with 3 rabbits in each group. Four sections with an area of 9 cm² each section on the back of each rabbit were shaved. The shampoo granules of Examples 1-5 and Comparative Examples 1-4 were each mixed with water to give a stick emulsion; 1 mL of each of the stick emulsions and the liquid shampoo of Comparative Example 8 was evenly applied on the shaved sections of three rabbits of the respective groups. The sections were covered with a transparent film for 4 h, then the sample was rinsed out, and residue of the sample on the section was removed by clean water. The test sections were observed for local skin reaction at 1, 24 and 48 h after removal of the sample.

Through the test, none of 39 tested rabbits showed local stimulation reactions such as skin erythema and edema, indicating that the shampoo granules prepared by this disclosure were safe and have no any irritation or adverse reaction.

### 3. Product's Sensory Performance Test:

The shampoo granules prepared in Examples 1-5 and Comparative Examples 1-7, and the liquid shampoo prepared in Comparative Example 8 were tested for their sensory performance.

130 subjects ranging from 20 to 60 years old were randomly selected, including 70 males and 60 females. The subjects were randomly divided into thirteen groups, with 10 persons in each group. The samples prepared in Examples 1-5 and Comparative Examples 1-8 were trialed respectively, and evaluated for usage experience. The samples were each trialed for 2 weeks. Based on their usage experience, the subjects evaluated the samples based on a scoring system. Evaluation items included cleansing and decontamination efficacy, easy rinsing without residue, gentle and moisturizing performances. A 10-point scale was adopted for scoring, 1 score means the worst and 10 scores mean the best. The evaluation results were represented by the average scores of all subjects in each group, as shown in Table 4. The higher the average score, the better the performance.

TABLE 4

| | Cleansing | Easy | | | | Easy | |
|---|---|---|---|---|---|---|---|
| | and | rinsing | Gentle and | | Cleansing and | rinsing | gentle and |
| | decontamination | without | moisturizing | | decontamination | without | moisturizing |
| Sample | efficacy | residue | performance | Sample | efficacy | residue | performance |
| Ex. 1 | 9.5 | 9.8 | 9.6 | CEx. 3 | 7.5 | 7.9 | 8.3 |
| Ex. 2 | 8.8 | 9.2 | 8.9 | CEx. 4 | 8.2 | 7.8 | 7.5 |
| Ex. 3 | 9.3 | 9.3 | 9.3 | CEx. 5 | 8.0 | 8.3 | 7.2 |
| Ex. 4 | 9.4 | 9.4 | 9.3 | CEx. 6 | 8.1 | 8.2 | 7.4 |
| Ex. 5 | 9.4 | 9.6 | 9.4 | CEx. 7 | 6.8 | 8.0 | 7.2 |
| CEx. 1 | 6.4 | 7.3 | 8.1 | CEx. 8 | 8.5 | 7.5 | 7.7 |
| CEx. 2 | 7.0 | 7.8 | 8.2 | | | | |

Note:
Ex. represents “Example”; CEx. represents “Comparative Example”.

As can be seen from the results in Table 4, the shampoo granules prepared in the examples of this disclosure have excellent gentle decontamination efficacy. The granular product is easy to rinse without residue, which can significantly reduce the amount of water when the granular product is used for washing hair. In particular, the shampoo granule prepared in Example 1 has the best effect. However, in Comparative Examples 1-2, sodium cocoyl hydroxyethylsulfonate or sodium lauryl glutamate is absent, and in Comparative Examples 3-4, the mass ratio of sodium cocoyl hydroxyethylsulfonate to sodium lauryl glutamate exceeds the range specified in this disclosure, thus the resulting shampoo granules have significantly reduced cleansing and decontamination efficacy compared with that of Example 1. It indicates that only when sodium cocoyl hydroxyethylsulfonate and sodium lauryl glutamate are blended as the surfactant in an optimized range specified in this disclosure, the resulting shampoo granules can achieve an excellent cleansing efficacy, and are easy to rinse without residue, thereby improving the consumer's experience. In Comparative Examples 5-6, two of the four softener components the Lactobacillus/rye flour ferment filtrate, the acanthus kernel oil, the grapeseed oil and the avocado fruit oil are absent, the gentle nourishing efficacy of shampoo granules is decreased. In Comparative Example 7, sodium lauryl glutamate, the Lactobacillus/rye flour ferment filtrate, and the acanthus kernel oil are absent, the comprehensive use effect of the shampoo granule is obviously reduced compared with that of Example 1, indicating that the specified surfactant and softener components in this disclosure cooperate and influence with one another, so that the resulting shampoo granule can achieve more gentle and excellent cleansing and decontamination efficacy. However, a liquid shampoo is directly prepared in Comparative Example 8, instead of a shampoo granule as described in this disclosure, the comprehensive efficacy is also reduced under the same components, amounts and conditions as those in Example 1. Therefore, the shampoo granule prepared by the specified method of this disclosure can effectively avoid degradation or inefficiency of active ingredients, thereby improving the performance and efficacy of the product.

### 4. Stability Test:

The shampoo granule prepared in Example 1 and the liquid shampoo prepared in Comparative Example 8 were tested for stability, and the test method was as follows.

The internal control standard used in this disclosure was higher than the Chinese national standards and light industry standards. According to the internal control standard, the sample was stored at a high temperature of 45±1° C. for 2 months, and at a low temperature of −18±1° C. for an additional 2 months; then the sample was subjected to a three-cycle high-low temperature test, each cycle consisting of 24-hour storage at a high temperature of 45±1° C. followed by 24-hour storage at a low temperature of −18±1° C. The results were shown in Table 5.

TABLE 5

| | | | | | Three-cycle |
|---|---|---|---|---|---|
| | | | | | high-low |
| | | | 45 ± | −18 ± | temperature |
| Sample | Test conditions | | 1° C. | 1° C. | test |
| Example 1 | Initial | Appearance | White | White | White |
| | | | granules | granules | granules |
| | | pH (an aqueous | 6-7 | | |
| | | solution of the | | | |
| | | sample with a | | | |
| | | weight ratio of | | | |
| | | the sample to | | | |
| | | water of 1:10) | | | |
| | | at room | | | |
| | | temperature | | | |
| | After test | | The granules showed no significant | | |
| | | | change. | | |
| | Conclusion | | Qualified | | |
| Compar- | Initial | Appearance | White | White | White |
| ative | | | emulsion | emulsion | emulsion |

| | | |
|---|---|---|
| Example 8 | pH (an aqueous | 6-7 |

solution of the
sample with a
weight ratio of
the sample to
water of 1:10)
at room
temperature

| | |
|---|---|
| After test | The emulsion had discoloration and |
| | slight stratification. |
| Conclusion | Unqualified |

As can be seen from the results in Table 5, the shampoo granules obtained in this disclosure have significant stability, and stably and fully exert their efficacy. However, the liquid shampoo of Comparative Example 8 shows discoloration and slight stratification under different test conditions, indicating poor stability. The above results indicate that by adopting a specified preparation method to transform a traditional liquid shampoo into a shampoo granule in this disclosure, the efficacy stability of the product can be effectively enhanced. The shampoo granule is also convenient to carry and transport, and offers significant advantages in multiple aspects such as environmental protection, economy, convenience and energy saving.

Finally, it should be noted that the above embodiments are intended to illustrate the technical solutions of the disclosure and not to limit the protection scope thereof. Although the disclosure is described in detail by reference to preferred embodiments, persons of ordinary skill in the art should understand that the technical solution of the disclosure can be modified or equivalently replaced without deviating from the substance and scope of the technical solution of the disclosure.

What is claimed is:

1. A shampoo granule, wherein the shampoo granule comprises the following components in parts by weight: 0.0008-0.0072 parts of a softener, 53-92 parts of a surfactant, 5-25 parts of an adsorbent, 1-10 parts of a moisturizer, 0.5-6 parts of an anti-caking agent, 0.4-5 parts of a hair regulator, 0.1-1 part of a pH regulator, 0.2-1.4 parts of a preservative, 0.1-1 part of a thickener and 0.5-1.5 parts of a fragrance;
the surfactant is a mixture of sodium cocoyl hydroxyethylsulfonate and sodium lauryl glutamate; and
the softener is a mixture of acanthus kernel oil, avocado fruit oil, grapeseed oil and Lactobacillus/rye flour ferment filtrate.

2. The shampoo granule according to claim 1, wherein the shampoo granule comprises the following components in parts by weight: 0.0015-0.0045 parts of the softener, 62-82 parts of the surfactant, 10-20 parts of the adsorbent, 3-8 parts of the moisturizer, 1-5 parts of the anti-caking agent, 1-3 parts of the hair regulator, 0.2-0.8 parts of the pH regulator, 0.4-1.2 parts of the preservative, 0.2-0.8 parts of the thickener and 0.7-1.3 parts of the fragrance.

3. The shampoo granule according to claim 2, wherein the shampoo granule comprises the following components in parts by weight: 0.004 parts of the softener, 75 parts of the surfactant, 20 parts of the adsorbent, 5 parts of the moisturizer, 3 parts of the anti-caking agent, 2 parts of the hair regulator, 0.5 parts of the pH regulator, 0.8 parts of the preservative, 0.5 parts of the thickener and 1 part of the fragrance.

4. The shampoo granule according to claim 1, wherein a mass ratio of sodium cocoyl hydroxyethylsulfonate to sodium lauryl glutamate is (57-67):(5-15).

5. The shampoo granule according to claim 4, wherein the mass ratio of sodium cocoyl hydroxyethylsulfonate to sodium lauryl glutamate is 60:15.

6. The shampoo granule according to claim 1, wherein a mass ratio of the acanthus kernel oil the avocado fruit oil:the grapeseed oil:the Lactobacillus/rye flour ferment filtrate is (0.0005-0.0015):(0.0005-0.0015):(0.0005-0.0015):(0.0005-0.0015).

7. The shampoo granule according to claim 1, wherein the adsorbent comprises a modified corn starch, the moisturizer comprises betaine, the hair regulator comprises guar gum hydroxypropyl trimethyl ammonium chloride, and the thickener comprises hydroxypropyl methyl cellulose.

8. The shampoo granule according to claim 1, wherein the anti-caking agent comprises magnesium stearate, the pH regulator comprises citric acid, the preservative comprises chlorphenesin and/or benzyl alcohol, and the fragrance comprises an essence.

9. The shampoo granule according to claim 1, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules within a 10-100 mesh range, to give the shampoo granule.

10. The shampoo granule according to claim 9, wherein in step S2, a mass ratio of the powder to the thickener aqueous solution is 1:0.3.

11. The shampoo granule according to claim 2, wherein the adsorbent comprises a modified corn starch, the moisturizer comprises betaine, the hair regulator comprises guar gum hydroxypropyl trimethyl ammonium chloride, and the thickener comprises hydroxypropyl methyl cellulose.

12. The shampoo granule according to claim 3, wherein the adsorbent comprises a modified corn starch, the moisturizer comprises betaine, the hair regulator comprises guar gum hydroxypropyl trimethyl ammonium chloride, and the thickener comprises hydroxypropyl methyl cellulose.

13. The shampoo granule according to claim 2, wherein the anti-caking agent comprises magnesium stearate, the pH regulator comprises citric acid, the preservative comprises chlorphenesin and/or benzyl alcohol, and the fragrance comprises an essence.

14. The shampoo granule according to claim 3, wherein the anti-caking agent comprises magnesium stearate, the pH regulator comprises citric acid, the preservative comprises chlorphenesin and/or benzyl alcohol, and the fragrance comprises an essence.

15. The shampoo granule according to claim 2, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules within a 10-100 mesh range, to give the shampoo granule.

16. The shampoo granule according to claim 3, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device, to give granules, and drying the granules at a high temperature; and
S3, sieving granules within a 10-100 mesh range, to give the shampoo granule.

17. The shampoo granule according to claim 4, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature;
S3, sieving granules within a 10-100 mesh range, to give the shampoo granule.

18. The shampoo granule according to claim 6, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules with a 10-100 mesh range, to give the shampoo granule.

19. The shampoo granule according to claim 7, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules with a 10-100 mesh range, to give the shampoo granule.

20. The shampoo granule according to claim 8, wherein the shampoo granule is prepared by a method comprising the following steps:
S1, mixing the surfactant, the adsorbent, the moisturizer, the anti-caking agent, the hair regulator, the fragrance, the pH regulator, the preservative and the softener evenly to give a mixture, then grinding the mixture into powder;
S2, mixing the thickener with water to give a thickener aqueous solution with a mass concentration of 0.4-0.6%, adding the powder obtained in step S1 to a granulator, then spraying the thickener aqueous solution into the granulator by a spraying device to give granules, and drying the granules at a high temperature; and
S3, sieving granules with a 10-100 mesh range, to give the shampoo granule.
